(19) United States
(12) Patent Application Publication
(10) Pub. No.: US 2008/0045925 A1
(43) Pub. Date: Feb. 21, 2008

(54) Drug delivery system

(76) Inventors: Matthew J. Stepovich, Santa Cruz, CA (US); Michael I. Falkel, Carmel Highlands, CA (US); Kenneth G. Davenport, Fort Collins, CO (US)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 11/820,208

(22) Filed: Jun. 18, 2007

Related U.S. Application Data
(60) Provisional application No. 60/814,296, filed on Jun. 19, 2006.

(51) Int. Cl. A61M 31/00; A61M 37/00; A61M 5/44

(52) U.S. Cl. 604/518; 604/113; 604/82

(57) ABSTRACT

System for the delivery of drugs from sealed cartridges provide for versatile and convenient delivery of the drugs. The system can be designed to deliver drugs from a plurality of cartridges with delivery through a manifold that connects to the plurality of cartridges. In some embodiments, the system mediates the heating of the drug such that it can be delivered at a temperature more closely approximating body temperature. In some embodiments, the composition of one cartridge is used to adjust the pH of the composition of the resulting mixture to achieve a desirable blended drug. The systems and procedures are particularly advantageous for the delivery of dental anesthetics.

### CROSS REFERENCE TO RELATED APPLICATIONS

This application claims priority to copending U.S. Provisional Application 60/814,296, with a filing date of Jun. 19, 2006, entitled “Method and Apparatus for Aggregating Carpules for use With an Anesthetic Pump,” incorporated herein by reference.

### FIELD OF THE INVENTION

The invention relates to powered drug delivery systems and methods for drug delivery, such as the delivery of dental anesthetics, using an automated system.

### BACKGROUND OF THE INVENTION

The hypodermic syringe has been an integral part of the medical and dental professions for long periods of time. Other delivery systems have been developed in the heath care industry for delivery of a range of drugs (chemical, biologic or other substances used in health care), such delivery systems including, for example, nebulizers, intravenous bottles, catheters and the like. In dentistry, one widely-used delivery system is comprised of a disposable pre-filled anesthetic cartridge that is loaded into the body of a hand held hypodermic syringe. The syringe is then used to inject the anesthetic from the cartridge into the patient's tissue.

### SUMMARY OF THE INVENTION

In a first aspect, the invention pertains to a method for the delivery of multiple units of a drug from sealed cartridges to a patient. The method comprises selectively delivering the drug from a plurality of individually sealed first cartridges using a motor to advance a plug within a cartridge to drive the drug from the cartridge. The first cartridges can be positioned on a stationary housing. The drug from the first cartridges flows through a manifold fluidly connected to flexible tubing, and the flexible tubing is fluidly connected to a delivery component that delivers the drug to the patient. Generally, the drug flows from the first cartridges to the delivery component through a sealed fluid path.

In another aspect, the invention pertains to a drug delivery system comprising a housing, a cartridge holder, a plurality of transfer tubes, a manifold, flexible tubing, and a patient delivery component. The housing can comprise at least one motor operably connected to a plurality of pistons. The cartridge holder can comprise a plurality of positioning slots configured to hold a fluid cartridge in an alignment to be engaged by one of the pistons when the piston is translated. The plurality of transfer tubes generally comprise a first end configured to engage and enter a sealed cartridge at a positioning slot. In some embodiments, at least one transfer tube comprises a displaceable cap covering the first end of the transfer tube. The manifold can comprise an output port and a plurality of channels in which a channel is fluidly connected with a transfer tube and in which the manifold has a configuration in which flows from the channels combine while having a fluid connection to the output port. The flexible tubing comprises a first end and a second end, in which the first end is fluidly connected to the output port of the manifold. The patient delivery component can comprise a fluid outlet, the patient delivery component being fluidly connected with the second end of the flexible tubing.

In further aspects, the invention pertains to a drug delivery system comprising a housing, a cartridge holder, at least one transfer tube, flexible tubing, a patient delivery component and a heating element. The housing can comprise at least one motor operably connected to at least one piston. The cartridge holder generally has at least one positioning slot configured to hold a cartridge. The at least one transfer tube can be configured to enter a sealed cartridge to establish a flow passage to an output port. The flexible tubing has a first end and a second end in which the first end if fluidly connected to the output port of the transfer tube. The patient delivery component comprises a fluid outlet, the patient delivery component being fluidly connected with the second end of the flexible tubing. The heating element is configured to heat at least one component of the system.

In other aspects, the invention pertains to a method for the delivery of a mixture of drugs to a patient. Specifically, the method comprises delivering a mixture of different drugs from a manifold connected to a plurality of cartridges comprising at least a first cartridge and a second cartridge. The cartridges contain different drugs and are positioned on a stationary housing. The delivery of the drugs comprises using a motor to move a plug within the first cartridge to drive the drug from the first cartridge to the manifold. The manifold generally is fluidly connected to flexible tubing, and the flexible tubing generally is fluidly connected to a delivery component to deliver the mixture.

### BRIEF DESCRIPTION OF THE DRAWINGS

FIG. 1 is a perspective view of an embodiment of a drug delivery system that can hold two cartridges for delivery with hidden structure shown in phantom lines.

FIG. 2 is a perspective view of the delivery system of FIG. 1 in which a cartridge holder is being placed onto a stationary housing at a docking position.

FIG. 3 is a perspective view of the stationary housing of the system of FIG. 1 with hidden structure shown in phantom lines.

FIG. 4 is a perspective view of the cartridge holder of FIG. 1 with two loaded cartridges.

FIG. 5 is a perspective view of the cartridge holder of FIG. 4 with a first cartridge being loaded into position.

FIG. 6 is a perspective view of the cartridge holder of FIG. 4 with a first cartridge loaded into position and a second cartridge in position for loading into the second position of the cartridge holder.

FIG. 7 is a perspective view of another embodiment of a drug delivery system with five cartridge positions within a cartridge holder.

FIG. 8 is a perspective view of the delivery system of FIG. 7 with the cartridge holder removed form the docking position in a stationary housing with hidden structure shown in phantom lines.

FIG. 9 is a perspective view of an alternative embodiment of a cartridge holder.

FIG. 10 is a perspective view of a drive system of the delivery system of FIG. 7 in which the drive system is separated from a housing to expose the components of the drive system.

FIG. 11 is an alternative perspective view of the drive system of FIG. 10.

FIG. 12 is a second alternative perspective view of the drive system of FIG. 10 in which the pistons are shown in an advanced position.

FIG. 13 is an exploded perspective view of a cartridge holder of the delivery system of FIG. 7.

FIG. 14 is a perspective view of the delivery system of FIG. 1 in which the pistons are advanced to a priming position.

FIG. 15 is a perspective view of the delivery system of FIG. 1 in which the pistons are shown in a position following a delivery of a majority of the drug from the cartridges.

FIG. 16 is a side view of a cartridge, cartridge slot and piston shown in different stages of the delivery process.

### DETAILED DESCRIPTION OF THE INVENTION

Delivery systems for drugs described herein provided for efficient and convenient delivery of drugs, such as anesthetics, based on versatile designs. In some embodiments, the system provides for the easy loading of multiple cartridges or carpules of a drug that can then be selectively delivered to a patient. The cartridges are generally held by a stationary base unit with a housing. The drugs from the cartridges can be combined for delivery through a flexible tube to a delivery element, such as a hand piece with a hypodermic needle. In additional or alternative embodiments, the base unit comprises one or more heating elements so that the cartridges, the fluid within the cartridges and/or other system components can be heated relative to room temperature so that the drug can be delivered to the patient at a temperature closer to body temperature. A motor can be used to move pistons or plungers to drive the drug from the cartridges for delivery to the patient. The systems and methods are particularly useful for anesthetizing dental patients.

In general, the apparatuses described herein can be used to deliver a drug or a combination of drugs to a patient. Patients generally can be humans, farm animals, pets or other mammals, although human patients are of particular interest. The drug can be any fluid, which flows appropriately for delivery. Thus, the system can deliver the drug, for example, for ingestion, inhalation or injection into a patient. In embodiments of particular interest, the drug and portions of the flow pathway from the cartridge to the delivery element along with corresponding portions of the apparatus are sterile. An injection can be, for example, subcutaneous, intravenous, intra-arterial, intradermal, or intramuscular, or for injection into bone or other soft tissue. Drugs can be, for example, medicinal/pharmacological compositions, nutrients, such as glucose, volumizing compositions, such as saline, or other beneficial fluids for delivery to the patient. A wide range of drugs are suitable for delivery using the techniques and equipment described herein. Procedures of particular interest include, for example, dental procedures, in which the apparatuses described herein can be used for the delivery of anesthetic into tissue within a patient's mouth.

Local anesthetics have been used for more than 100 years to limit or eliminate pain associated with dental procedures. A conventional system comprises a reusable stainless steel hand held syringe with a disposable needle and a disposable carpule or cartridge. Carpule is a term often used for cartridges of dental anesthetic, and for the purposes herein this term is used interchangeably with cartridge unless otherwise noted. Generally, a drug cartridge can comprises a glass or plastic tube with a penetrable cap covering one end and a slidably mounted rubber plug located inside the tube at its other end, the cap and the plug creating a fluid tight chamber for the drug. In a typical dental anesthetic cartridge, this chamber contains 1.8 cubic centimeters (cc) of liquid anesthetic.

With a dental syringe, the disposable needle is attached to the stainless steel syringe housing, part of the needle extends into the syringe housing to form a transfer tube. When the anesthetic cartridge is loaded into the syringe, the transfer tube pierces the cartridge's penetrable cap, establishing a fluid path between the contents of the cartridge and the hypodermic needle. Once the cartridge is loaded in the syringe housing and the transfer tube has pierced the penetrable cap, the practitioner uses his or her thumb to press a piston in the syringe housing forward into the slidably mounted rubber plug, where the piston engages the plug with a small harpoon. The practitioner now places forward pressure on the piston to expel a small amount of fluid from the system, purging any air bubbles. At that point, the system is loaded and primed. The needle is then inserted into the patient's tissue, and the piston is pulled in reverse to place negative pressure on the system, which in turn draws a small amount of fluid from the patient's tissue back into the cartridge, where it can be visually inspected for the presence of blood, which would indicate that the hypodermic needle is in a blood vessel. The process of using negative pressure to draw a small amount of fluid from the patient's tissue into the system is aspiration. If no blood is aspirated, the injection process is continued, generally until the cartridge is exhausted.

In many dental procedures, it is desirable to use more than cartridge of anesthetic to achieve a desired degree of analgesia. Using the conventional syringe, the entire loading, priming and aspiration process needs to be repeated once a cartridge is replaced. Thus, a significant amount of time is expended in the process of replacing the cartridge for delivering the desired amount of anesthetic. Some procedures use two, three or more such reloads.

While proper technique for needle insertion can reduce the pain from the process, pain can result from delivery of the anesthetic at a temperature significantly different from body temperature. To reduce this pain, it can be desirable to warm the anesthetic before it is injected. In some embodiments, the apparatuses described herein provide for heating the drug above room temperature before it reaches the patient's tissue. In some embodiments, components of the delivery system can be warmed so that the drug reaches the patient in a desirable warmed state.

In some embodiments, the delivery systems herein provides for the delivery of fluids from a plurality of cartridges that can be delivered sequentially, simultaneously, or at varying rates relative to each other, such that a health care professional can select delivery with certain desired characteristics. For example, the ability to deliver drugs from more than one cartridge in this manner provides for the delivery of a flexible volume of a particular drug, based on the total number of cartridges of the drug loaded into the system, without the need for a reload.

In some embodiments, the system also provides for the delivery of different drugs in a sequence that can be controlled by the practitioner, such as one cartridge following the next. In additional or alternative embodiments, the system allows the practitioner to stop and start delivery of drugs from any cartridge at the practitioner's election, whether or not that cartridge had been completely exhausted. Such flexibility allows the practitioner to end the delivery of one drug, as circumstances might dictate, and then restart delivery of that drug or begin the delivery of another drug when other conditions dictate.

Also, in some embodiments, the system allows the practitioner to deliver drugs from more than one cartridge at the same time, in order to deliver a mixed composition of different drugs from different cartridges, the mixed composition having different properties than the drug contained in of any one cartridge. In additional or alternative embodiments, the system allows the practitioner to control the ratio of drugs delivered from more than one cartridge by, among other things, varying the speeds of the pistons relative to each other, to achieve specific desired mixtures of drugs.

The ability to mix the fluids from different cartridges provides for the delivery of additional amounts of a particular composition, for the sequential delivery of different compositions that provide a corresponding combination of benefits, and/or for the delivery of a mixed composition in which the different compositions mix to form a mixed composition that has different properties than the individual compositions, for instance to deliver pH adjusted anesthetic fluid, or warmed and pH adjusted anesthetic fluid. Although the apparatuses and methods are more generally applicable, the description herein generally provides additional details with respect to dental anesthetic delivery, and a person of ordinary skill in the art can adapt appropriate portions of this discussion with respect to other applications based on the disclosure herein.

Apparatuses of particular interest comprise a housing with at least one motor or the like, along with a cartridge holder connected to flexible tubing, and a delivery element connected to the flexible tubing. The motor is connected to a drive element that engages a cartridge loaded onto the cartridge holder such that it can control delivery of fluid from the cartridge. The apparatus can comprise a manifold, and the cartridge holder can have positions for a plurality of cartridges such that the plurality of cartridges all feed into the flexible tubing through the manifold. In this way, fluids from a plurality of cartridges can be sequentially and/or simultaneously delivered through the tubing. The cartridge holder generally comprises a transfer tube that interfaces with a cartridge to establish a fluid connection between the transfer tube and the contents of the cartridge. The delivery element can be any suitable device to deliver a drug to a patient generally in sterile condition. A suitable delivery element can be for example, a handpiece with a hypodermic needle, an infuser, a nebulizer, a catheter or other apparatuses or combination of apparatuses that deliver the drugs from the flexible tubing to the patient.

For embodiments that can support a plurality of cartridges, the delivery of fluid from the set of cartridges can be simultaneously controlled by one motor to deliver simultaneously fluid from all loaded cartridges. In alternative embodiments, the delivery of fluid from one or more different cartridges can be separately controlled. The separate control of delivery from different cartridges can be effectuated through the use of separate motors and/or drive elements for the distinct cartridges, although in some embodiments, the contents of some cartridges are simultaneously delivered while the contents of one or more other cartridges is separately controlled. Alternatively or additionally, a transmission can be used such that one motor can be used to independently control the delivery of the contents of a plurality of cartridges.

If there is a manifold, a plurality of transfer tubes lead to the manifold. If a cartridge is loaded onto each transfer tube, then the contents of the cartridges are combined in the manifold, and the system generally is fluid tight from the cartridge(s) through the delivery element. However, in some embodiments, the system remains fluid tight even with one or more slots in the cartridge holder “open” or not loaded with a cartridge. This ability provides the system the flexibility to function fully or partially loaded.

In some embodiments, the system may comprise a flow control mechanism that blocks the open end of one or more transfer tubes, maintaining the fluid integrity of the system even when on or more slots in the cartridge holder remain open or unloaded. Flow control elements can include, for example, valves, such as check valves, caps and tip covers. In an embodiment of the system, a tip cover comprised of rubber or another soft flexible material is pressed onto the open end of one or more transfer tubes. The tip cover prevents leakage from the transfer tube into an open slot. However, during the loading procedure, the tip cover can be removed from the end of the transfer tube for any slot in which a cartridge is loaded. Alternatively, in some embodiments, during cartridge loading, the drug cartridge's penetrable, e.g., puncturable, cap is pressed down toward the end of the transfer tube and makes contact with the tip cover. The penetrable cap then drives the tip cover down the transfer tube so that the tip cover no longer covers the end of the transfer tube, and with further downward pressure on the cartridge the transfer tube drives through or past the penetrable cap and into the interior of the drug cartridge. In this manner, the tip cover is moved out of its position sealing the tip of the transfer tube, which can then establish a fluid connection to the drug cartridge.

In some embodiments, the system can comprise one or more heating elements, such as electrical resistance heaters. The heating elements can be selected to interface with the cartridge holders to heat the contents of the cartridges prior to use. Additionally or alternatively, the system can comprise a platform, holster or other structure that engages the flexible tube and/or the delivery component such that these parts of the system can be appropriately heated prior to use. Systems with one or more heating elements may or may not further comprise one or more heat sensors to facilitate control of the heating process and/or contact or proximity sensors to identify slots in which cartridges have been inserted so that, among other things, only occupied slots may be heated. In embodiments without a temperature sensor, the heating process may be controlled through the selection of the temperature of the heating element and the time that the heating element is operated.

In general, the cartridges can hold any drug that has characteristics of a fluid. Thus, the drug may be a liquid, flowable powder, a dispersion, an aerosol, a colloid, a gas or the like. In some embodiments, a drug can be a medicinal composition, such as analgesics, anti-inflammatories, antibiotics, antiseptics, anesthetics, vasoconstrictors, combinations thereof or the like. Dental anesthetics include, for example, novocaine, lidocaine, prilocaine, mepivicaine and combinations thereof as well as anesthetics mixed with vasoconstrictors, like epinephrine. In some embodiments, a drug in one state, such as a liquid, may be combined with the same or a different drug in a different state, such as a powder or a gas. In some embodiments of particular interest, the drug comprises a liquid anesthetic, or a mixture of a liquid anesthetic and an alkalinizing substance.

The system can comprise a controller that mediates the delivery of the fluid according to appropriate instructions. The controller can comprise one or more displays, one or more input devices, volatile and/or non-volatile memory, a microprocessor or the like, appropriate electrical connections and a power supply. The controller can be located in the housing with the motor or in a separate housing. If the controller is located in a separate housing, the controller can be connected to the motor housing with a wired or a wireless connection, such as a blue tooth connection or other appropriate wireless technology. The motor correspondingly can have a suitable power supply and switches to control its function in cooperation with the controller, if any. The direct delivery of the fluid from the cartridge can be controlled by a user operated switch, which can be mounted, for example, on a hand piece, on a foot pedal or the like, using a wired or a wireless connection.

The system may comprise sensors and other mechanisms that detect the number of slots that are loaded with a cartridge (or that are not loaded), which slots are loaded or unloaded, or the type of drug that may be loaded in a slot. The system may use this information to calibrate the rate of flow to the patient, the dosage of the drug being delivered, the mix of drugs being delivered, or the relative amount of the drugs from each cartridge being delivered. The presence of a cartridge can be detected using a contact switch or the like, or through an electrical /electromagnetic or optical measurement. For example, the cartridge can include a radio frequency identification element, RFID, which are commonly used, which can supply information on the drug within the cartridge as well as other information, such as the volume of the cartridge. Similarly, optically readable information can be placed on the side of the cartridge, such as in the form of characters, bar codes or the like. Suitable readers are readily available to read coded information from the cartridge. The cartridge and slot can be shaped to have the cartridge loaded in a particular configuration to facilitate the reading of information. Similarly, the size and shape of cartridges and slots can be selected to limit the placement of cartridges within certain slots for the cartridge.

To perform a selected procedure, the health care professional selects the drug(s) to be delivered and the appropriate number of cartridges of such drug(s). The cartridge(s) are loaded into the cartridge holder. In embodiments in which the cartridge holder is separate from the system housing, the cartridge holder is loaded into position in contact with the system housing. In some embodiments, the user instructs the system with respect to the dosage to be delivered, the rate of flow during delivery, whether or not warming is to be used, and/or the contents of the cartridges. The user instructs the system to proceed, and the system delivers the drug(s) in a selected way upon commencement of the procedure.

In some embodiments, such as dental anesthetic embodiments, it can be desirable to adjust the pH of a drug prior to its delivery to a patient. To accomplish this pH adjustment, one cartridge can comprise an acidifying or an alkalinizing substance, such as acetic acid, or sodium bicarbonate. The controller may mediate the delivery of a desired amount of pH adjusting composition into the manifold. In general, it may be desirable to adjust the pH such that the system delivers a drug at or closer to the patient's physiological pH. For dental anesthetics, having a more physiological pH may improve the efficacy of the anesthetic, the pain experienced during injection, the onset time for the anesthetic and/or recovery time. The pH of anesthetic in commercially available dental cartridges is generally more acidic than physiological pH, the acidity extending the shelf life of the anesthetic cartridge. In dentistry, it may be useful to alkalinize the anesthetic near in time to the delivery of the alkalinizing anesthetic.

Once the health care professional and patient are ready for the procedure, the health care professional can depress a switch, such as a foot switch, to initiate the procedure. Similarly, the switch can be used to signal a pause in the procedure and/or a change in procedural steps, such as a change to an aspiration step and/or a change to a more rapid delivery step. For dental procedures, it can be desirable to have an initial slow delivery step as well as an aspiration step near the beginning of the procedure, and these steps can be regulated by the controller. Once initial steps are completed and a small amount of anesthetic has been delivered, a more rapid delivery step can be used. In alternative embodiments, the controller manages one or more of these procedural transitions.

Once the procedure is complete, appropriate cleaning procedures are followed to avoid the spread of any pathogens or other contaminants between patients. In general, any portion of the system in the body fluid pathway should be discarded or sterilized. In general, it can be cost and time effective to use appropriate disposable components. Thus, in some embodiments, the delivery component, the flexible tubing, the used cartridges, manifold and the cartridge holder are disposable. The housing can be designed for sterilization appropriate for instruments that are not on the fluid pathway.

The systems and procedures described herein offer advantages for health care professionals using the system. For example, multiple cartridges can be administered to a patient without delays associated with reloading the system. The system can be programmed to automate delivery related tasks that generally require the practitioner's efforts, such as providing the motive force for, and controlling the rate of, flow of the drugs to the patient, which may make the results more uniform and predictable and can free the practitioner to attend to other elements of the procedure. In some embodiments, the system's ability to combine compositions and to calibrate the combination provides for delivery of a desired composition in an easy and versatile way, such as at a desirable pH. Furthermore, heating of the drug can provide for more comfortable delivery and may increase the effectiveness of the drug.

Delivery Apparatus

The delivery systems described herein generally have a base unit connected via flexible tubing to a delivery component, which can be manipulated by the health care provider to deliver the drug to the patient. The drugs are generally provided in cartridges, which can be generally cylindrical with a sealed cap on one end that can be breached and a slideable plug or the like inside the tube that can be contacted through the other end of the tube. The cap can be penetrated by an element of the system to allow flow from the cartridge into portions of the system when pressure is placed on the slideable plug. One or more cartridges can be loaded into a base unit. The base unit can control the delivery of the drug using a motorized drive that engages the cartridge. The base unit can further comprise one or more heating elements to heat the cartridges and/or other components of the system. The cartridges can have different contents, and the physical parameters and/or visible markings of the cartridges may be altered depending on the contents to facilitate proper selection and placement of the cartridges in the system, and/or the system's recognition of a cartridge's contents.

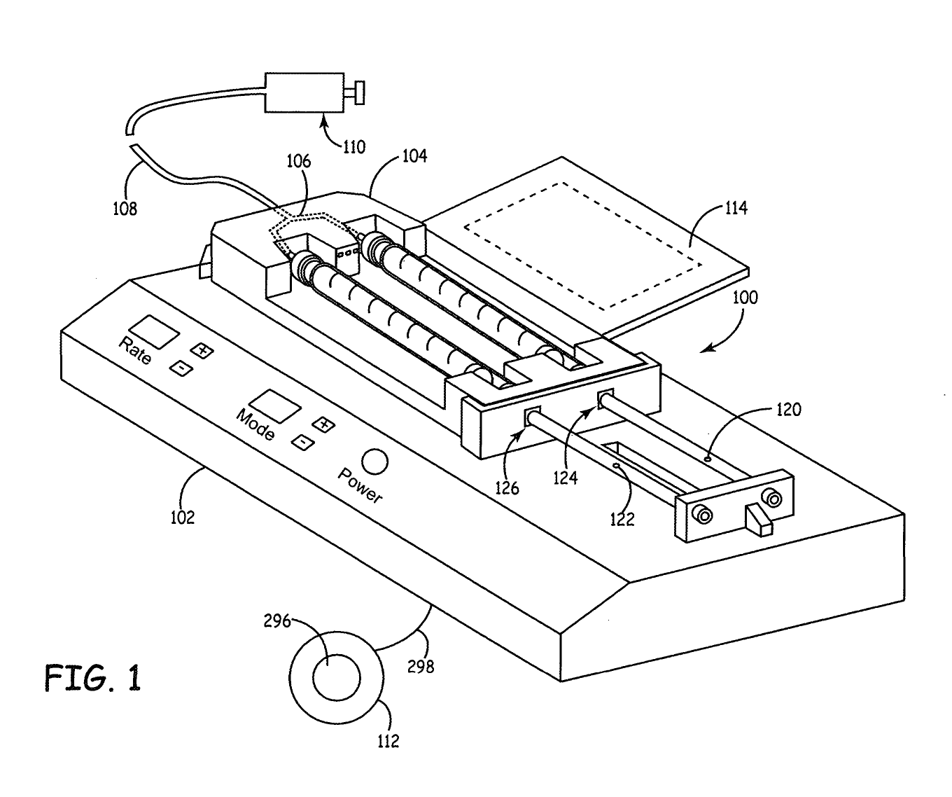

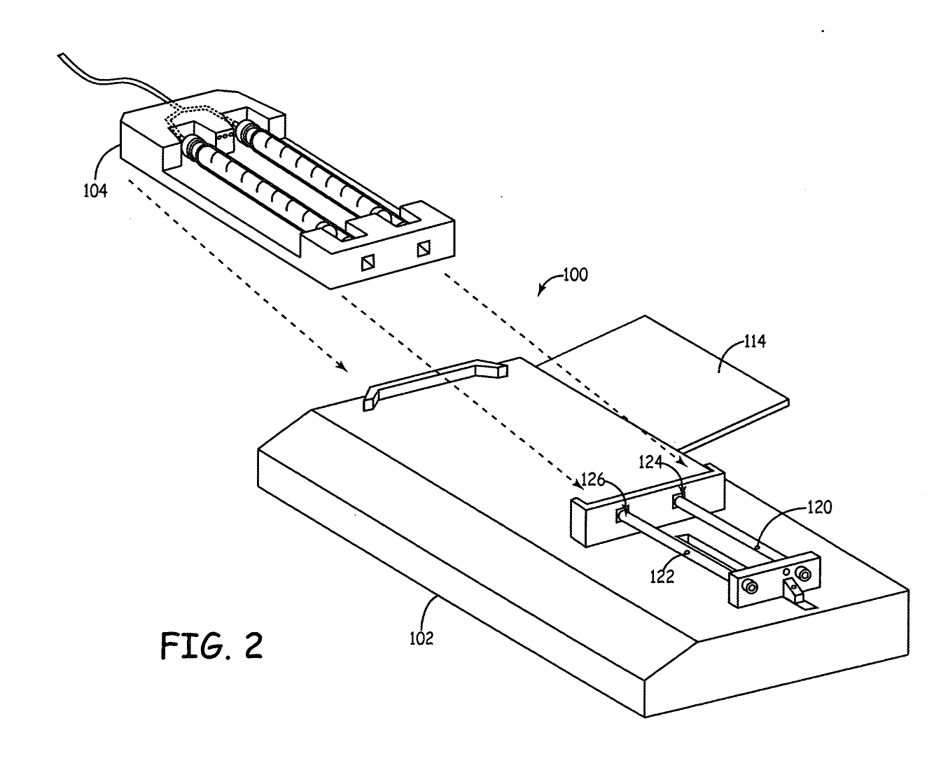

FIG. 1 depicts a first embodiment of a drug delivery system. As shown in FIG. 1, delivery system 100 comprises a stationary housing 102, a cartridge holder 104, a manifold 106, flexible tubing 108, a delivery component 110, positionable switch 112 and tubing heating structure 114. As shown in FIG. 1, cartridge holder 104 is located in the seated position on stationary housing 102. Note that in this illustration, pistons 120, 122 have not yet been driven forward through piston windows 124, 126 to engage the cartridges. FIG. 2 shows an exploded view with cartridge holder 104 positioned for placement onto stationary housing 102.

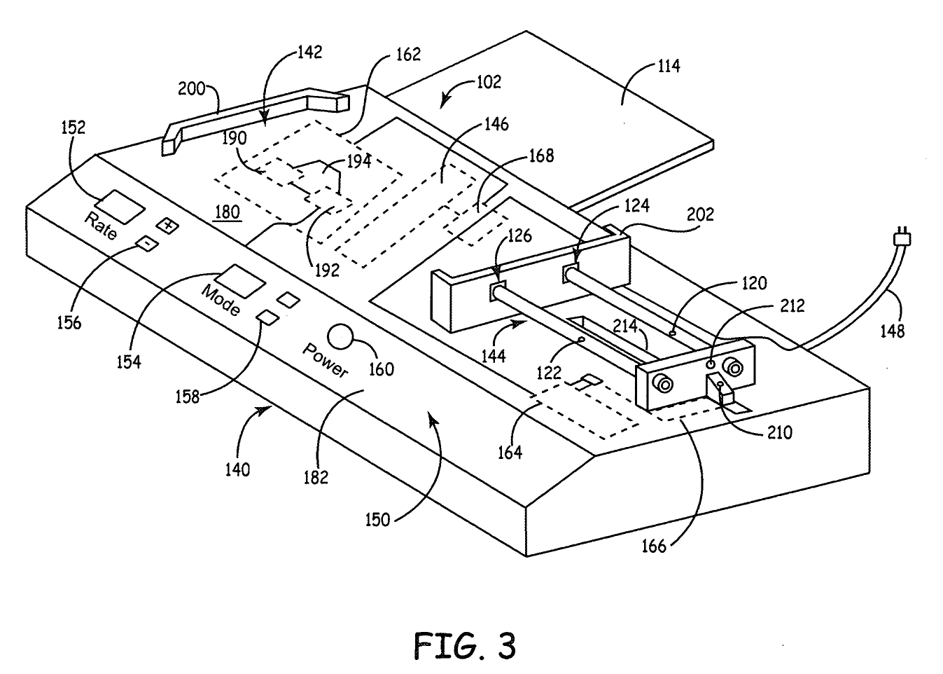

A view of stationary housing 102 separate from the cartridge holder is shown in FIG. 3. In this embodiment, stationary housing 102 comprises housing 140, docking station 142, drive system 144, heating element 146 and power cord 148. Housing 140 comprises cover 150, displays 152, 154, input controls 156, 158, power switch 160, controller 162, motor 164, transmission 166 and power supply 168. Cover 150 comprises a top surface 180 that supports docking station 142, a control surface 182 angled to facilitate viewing from the front of the device and suitable side walls and bottom. The components of cover 150 can be formed form any suitable materials, such as steel, aluminum or other metals or alloys, plastics, such as polycarbonates, fiberglass, composites or combinations thereof.

Displays 152, 154 can comprise any suitable display elements, such as liquid crystal elements, light emitting diodes or other elements including, for example, presently available and later developed commercial display elements. Drivers for displays 152, 154 can be part of controller 162 or separately located within cover 150. Input controls 156, 158 can be one or more buttons, touch pads, knobs, switches or other suitable input elements. Switch 160 can be any reasonable switch. While this embodiment is shown with two displays, two input elements and a switch, in general, the device can comprise one display or more then two displays and other numbers of input elements to provide a desired level of functionality and ease of use, and a person of ordinary skill in the art can provide selected elements to also provide desired appearance.

Controller 162 can comprise a microprocessor 190, display drivers 192, a bus 194 and other electronic components selected by design choice. Suitable components can be selected by a person of ordinary skill in the art to provide the selected functionalities. In some embodiments, controller 162 as well as some or all of the selected displays and input elements can be associated with a second housing physically separate from housing 102, which can then communicate with housing 104 to control motor 164 using wired or wireless communication.

Motor 164 can generally be any suitable motor. Suitable motors can comprise, for example, conventional induction motors, stepper motors, servo-motors, or the like. Suitable motors can be linear motors such that no transmission may be needed. If a transmission is used, transmission 166 can comprise, for example, a worm drive to convert rotational motion into linear actuation. Linear actuators are described further, for example, in U.S. Pat. No. 6,794,779 to Ma et al., entitled “Compact Electromechanical Linear Actuator,” and U.S. Pat. No. 5,557,154 to Erhart, entitled “Linear Actuator With Feedback Position Sensor Device,” both of which are incorporated herein by reference. A variable speed actuator is described, for example, in U.S. Pat. No. 4,970,861 to Randall, entitled “Geared Rotary-to-Linear Motion Converting System for Bidirectional Pump Drive,” incorporated herein by reference. In some embodiments, the system can comprise a plurality of motors, in which a particular motor drives the delivery of fluid from one or more cartridges such that flow from different cartridges can be at different rates and/or sequentially delivered. The system may account for the types of drugs contained in each cartridge and automate their delivery relative to the other cartridges to obtain a desired result, for instance using a cartridge of 8% sodium bicarbonate as an alkalizing agent for lidocaine, lidocaine with epinephrine, or mepivicane, each of the three having a unique pH that would require mixing with more or less sodium bicarbonate to achieve a physiologic pH.

Power supply 168 can be connected to power cord 148 to bring power into the system. Power supply 168 can comprise suitable transformers and the like to provide appropriate power for the motor, displays, controller elements and any other powered components. Components of power supply 168 can be distributed through the interior of housing 102 as desired to appropriately supply power to the appropriate components. In alternative or additional embodiments, power cord 148 can be replaced or supplemented with one or more batteries, which can be rechargeable batteries, and/or fuel cells. Docking station 142 can generally have any reasonable structure to support cartridge holder 104. Thus, the design of docking station 142 generally is correlated with the design of cartridge holder 104 such that they can appropriately interface. As shown in FIG. 3, docking station 142 comprises a first edge support 200 and second edge support 202, between which a cartridge holder can be placed and supported during drug delivery.

Drive system 144 applies movement of transmission 166 to one more cartridges to deliver fluid from the cartridge(s). As shown in FIG. 3, transmission 166 is operably connected to arm 210 that is attached to plate 212. Arm 210 translates within slot 214. Pistons 120, 122 are bolted to plate 212 such that movement of arm 210 correspondingly moves pistons 120, 122. Pistons 120, 122 pass through piston windows 124, 126, respectively, such that movement of arm 210 corresponds with linear motion of pistons 120, 122.

Heating element 146 can be a resistive heating element or other suitable structure. Heating element 146 can be electrically connected to power supply 168 and controller 162. Generally, heating element is placed under a material that provides at least reasonable thermal conductivity such that heat from heating element 146 can be transferred to cartridges loaded onto cartridge holder 104.

Power cord 148 can be selected to provide a desired amount of amperage to the system. In alternative embodiments, one or more batteries or fuel cells can be used alternatively or in addition to the power cord. Suitable batteries include, for example, rechargeable batteries. In other embodiments, stationary housing 140 may contain one or more rechargeable batteries, and may be removably coupled to a charging base (not shown) that has a power cord, allowing the user to decouple and use the system in a location away from the charging base after charging and/or warming is complete.

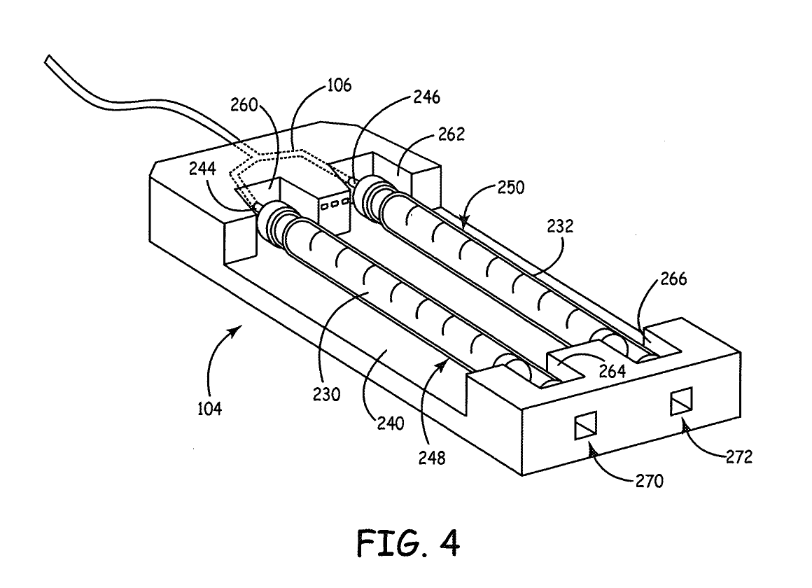

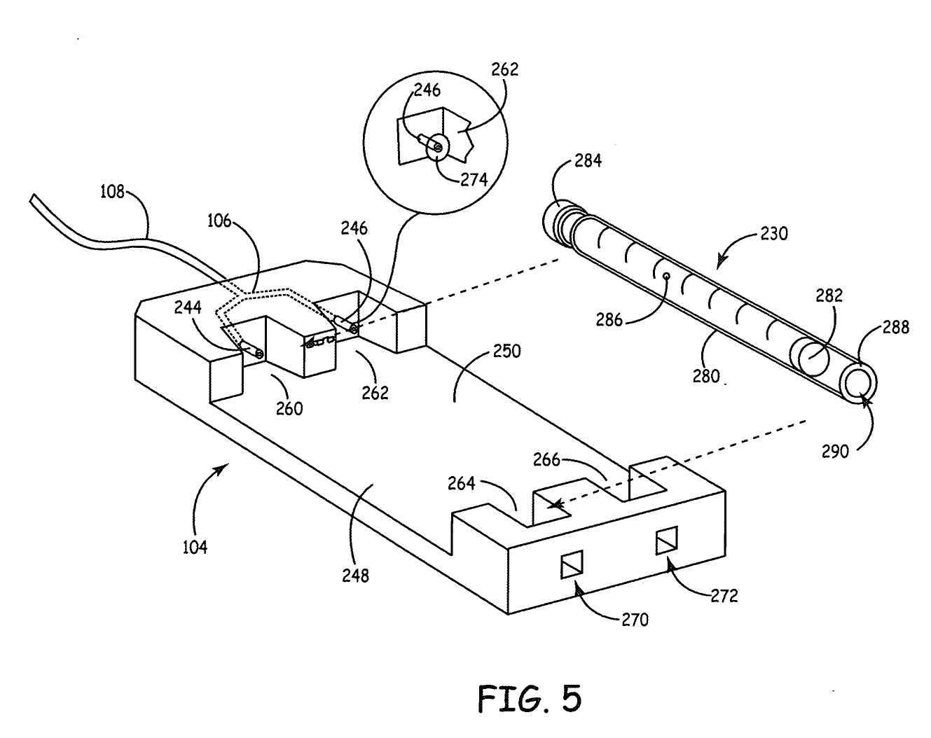

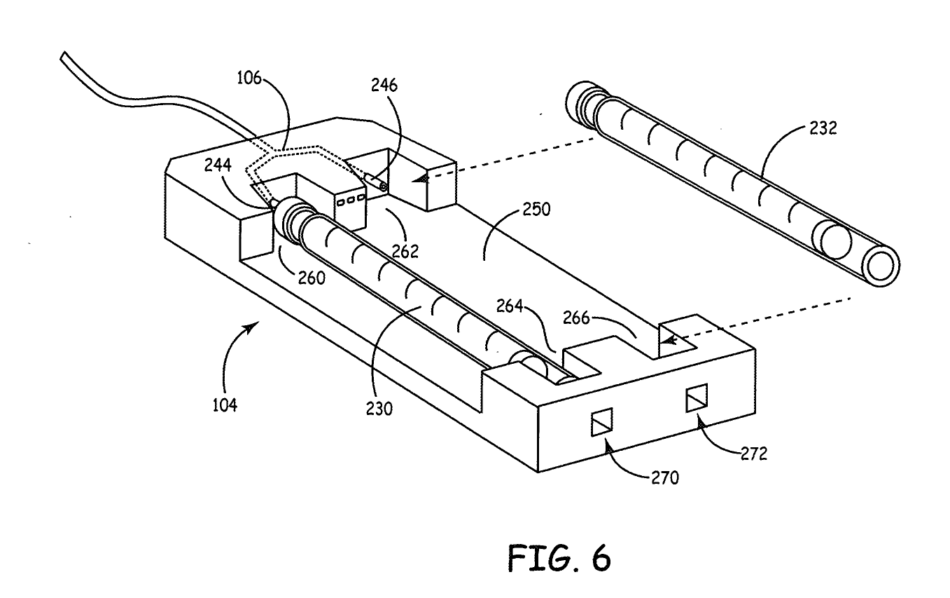

Cartridge holder 104 is depicted in FIGS. 4-6. In FIG. 4, cartridge holder 104 is shown loaded with two cartridges 230, 232, while in FIG. 5, cartridge holder 104 is shown with cartridge 230 positioned for loading and in FIG. 6, cartridge 230 is loaded and cartridge 232 is positioned for loading. Cartridge holder 104 comprises frame 240 and transfer tubes 244, 246. In this embodiment, manifold 106 is embedded within cartridge holder 104. Frame 240 has docking positions 248, 250 for respective fluid cartridges. As shown in FIGS. 4-6, docking positions 248, 250 are formed by first end slots 260, 262 and second end slots 264, 266. A variety of other configurations can be used for docking positions, such as one embodiment described further below as well as indentations or slots that extend over the length of the cartridge. Transfer tubes 244, 246 are configured to extend within first end slots to engage a cartridge with an end positioned within first end slots 260, 264. Second end slots 264, 266 comprise drive windows 270, 272 to provide for an interface of drive elements with loaded cartridges.

Referring to FIG. 5 and the insert, tip cover 274 covers the end of transfer tube 246 in this embodiment. Tip cover 274 blocks flow in to or out from transfer tube 246 unless and until tip cover 274 is disengaged from the opening of transfer tube 246 such as through removal or displacement further down transfer tube 246. For example, transfer tube 246 can have a plastic cap that is manually removed prior to loading a cartridge. In some embodiments, tip cover 274 comprises a polymer plug that can be punctured and displaced when pushed laterally along transfer tube 246 when loading a cartridge. Displacement of tip cover 274 along transfer tube 246 can be mediated by a spring or other elastic material that can be compressed when a cartridge is loaded and tip cover 274 is displaced.

Transfer tubes 244, 246 are generally designed to engage or break a seal into a sealed cartridge thereby establishing a fluid connection with the cartridge. In some embodiments, transfer tubes 244, 246 comprise a rigid plastic that may or may not have a sharp tip. Transfer tubes 244, 246 can pierce a seal on a cartridge, push open a seal member or otherwise appropriately engage a cartridge to break a seal and establish a fluid connection to the contents of the cartridge.

Referring to FIG. 5, cartridge 230 comprises a generally cylindrical tube 280, a slidably mounted plug 282, a penetrable cap 284 and a drug 286. Tube 280 can be made, for example, from glass, plastic or other suitable material that is compatible, e.g., inert, with respect to drug 286. Tube 280 generally has an open end 288 leading into channel 290 that provides access to slidably mounted plug 282. Slidably mounted plug 282 can be made, for example, from natural or synthetic rubber, other polymers that provide for desired mechanical and chemical properties or the like for engaging and making a slideable seal with tube 280. Penetrable cap 284 can comprise, for example, a rubber seal or the like that can be punctured, a polymer seal that can be displaced to open the seal upon interaction with a transfer tube or other appropriate seal that can be opened with a transfer tube.

As shown in FIGS. 1 and 4-6, manifold 106 is embedded within cartridge holder 104. In additional or alternative embodiments, transfer tubes 244, 246 lead to separate output ports that are fluidly connected to a separate manifold. For example, a manifold can be formed from rubber or other polymeric material so that there is a flexible manifold adjacent the cartridge holder. In embodiments in which the cartridge holder is designed to hold more than two cartridges, a first manifold configured to combine flows from a plurality of cartridges can interface with a second manifold that combines flow from additional cartridges separate from the cartridge holder.

Referring to FIG. 1, flexible tubing 108 can be formed from any suitable flexible polymeric material, such as natural or synthetic rubber Teflon®, or the like. Generally, the material is selected to be inert with respect to a range of drugs. Flexible tubing 108 generally can be selected to have any reasonable length and diameter. With respect to length, in many embodiments, reasonable lengths can be about 0.5 meters to about three meters in length, although any other reasonable length can be used.

Delivery component 110 can be configured to facilitate delivery of the drug to the patient in various ways, including, for example, via a hypodermic needle, via a catheter, oral delivery or inhaled delivery. Delivery component 110 can comprise a Leur fitting or other fitting to provide for connection to an intravenous delivery system or the like. In other embodiments, delivery component 110 can comprise a hand piece and a hypodermic needle for injection of the drug. For oral delivery, delivery component can comprise a rigid polymer element that can be conveniently directed to aim the delivery of the drug. For inhaled delivery, delivery component can comprise a mouth piece or the like such that the patient can conveniently inhale the drug as it is administered, or an atomizer or nebulizer. A specific embodiment of a delivery component 110 for an injection delivery is described further below. Also, hand pieces suitable for use in injection delivery are described further in copending U.S. provisional patent application Ser. No. 60/849,643 filed on Oct. 6, 2006 to Falkel et al., entitled “Method and Apparatus for Delivering Anesthetic,” incorporated herein by reference.

In some embodiments, delivery system 100 comprises positionable switch 112, which generally can be moved to a convenient location relative to stationary housing 102. Positionable switch 112 can comprise a simple structure to provide effectively binary type commands to stationary housing 102, although more complex switch structures can be used. As shown in FIG. 1, positionable switch 112 comprises a single button 296 and wire connection 298 that connects switch 112 with base unit 102. In alternative embodiments, positionable switch 112 has a wireless communication ability to interface with a base station, controller or other system component. Positionable switch 112 can be a foot switch, a hand operated switch or the like. In general, stationary housing 102 can be operated without a positionable switch, although in appropriate embodiments a positionable switch can be used to provide simple control without interacting directly with stationary housing 102.

In some embodiments, delivery apparatus 100 comprises tube heating structure 114. As shown in FIGS. 1-3, heating structure 114 is physically connected with stationary housing 102, although in other embodiments, heating structure 114 can be physically separate from stationary housing 102. Similarly, heating structure may or may not be supplied with power from power supply 168 or from a separate power supply, and similarly, heating structure may or may not have a separate controller from stationary housing 102. Tubing heating structure 114 generally can be designed to heat flexible tubing 108 and/or delivery component 110 such that the drug is heated for delivery and/or does not cool an undesirable amount for delivery. As shown in FIGS. 1-3, heating structure 114 is configured as a tray, but in other embodiments, heating structure 114 can be configured as a holster into which the tubing is placed prior to use, or other convenient structure.

As shown in FIGS. 1-6, delivery system 100 is configured for controlling the delivery simultaneously of at most two cartridges. In other embodiments, the system can be configured to facilitate the delivery of only one cartridge, although in other embodiments, the system is configured to hold a plurality of cartridges, such as three, four, five, six or more than six.

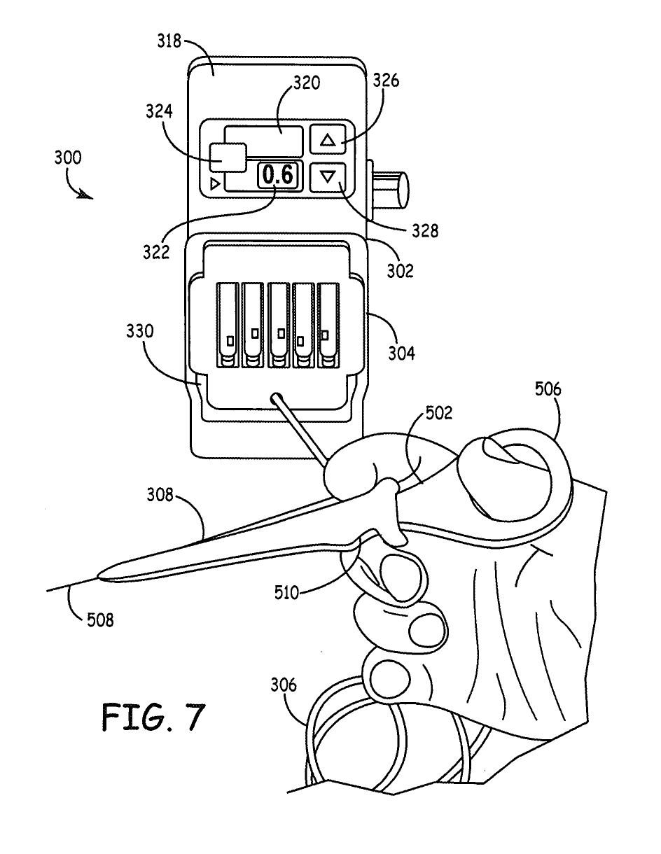

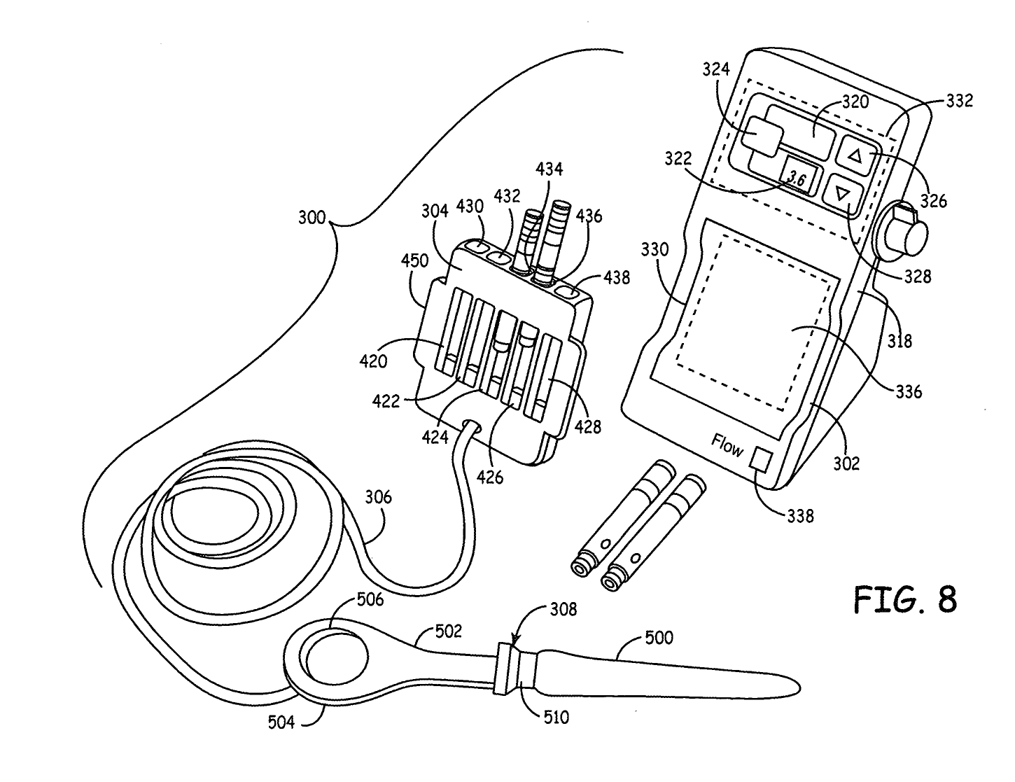

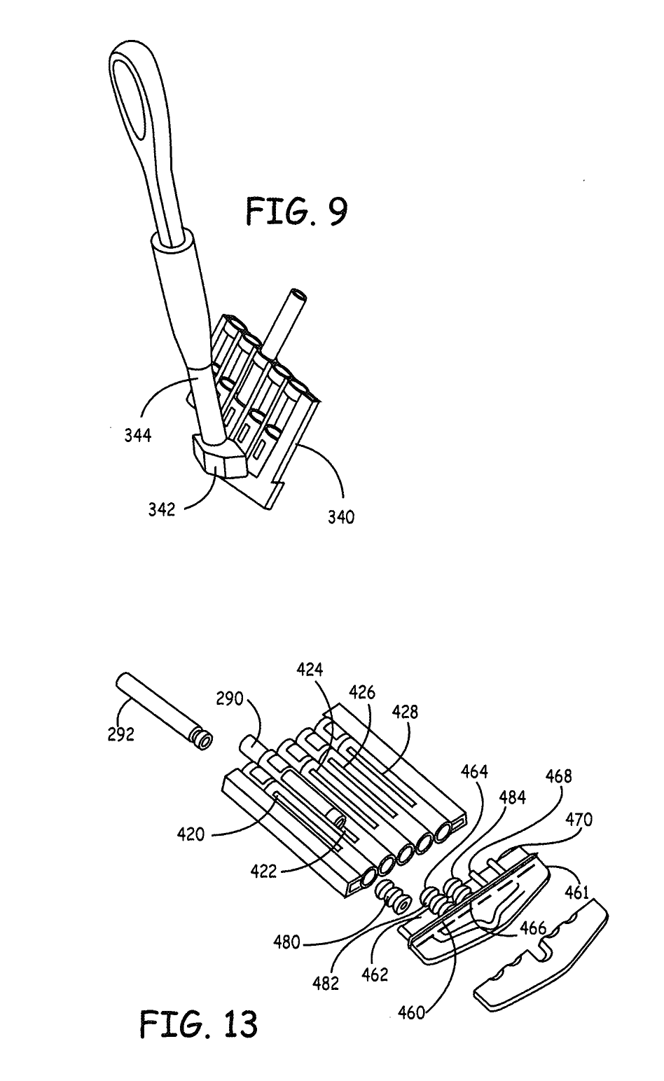

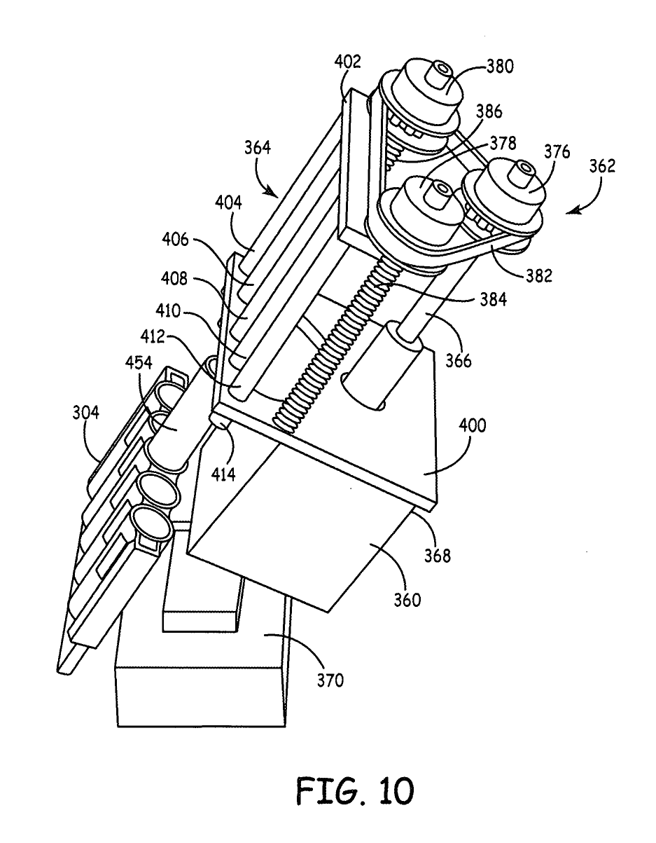

Another embodiment of a delivery system is shown in FIGS. 7 and 8. In this embodiment, delivery system 300 comprises a stationary housing 302, a removable cartridge holder 304, flexible tubing 306 and hand piece 308. In this embodiment, stationary housing 302 comprises housing 318, displays 320, 322, input pads 324, 326, 328, docking section 330, controller 332, drive unit 334 (FIGS. 10-12) heater 336 and heater on-off switch 338. Display 320 comprises three separate elements that can light to indicate flow at one of three speeds, slow, medium, or fast, and display 322 comprises a two digit display to depict the dose the practitioner elects to deliver. After delivery has begun, display 322 may transition to a mode where it displays the running amount of drug that has actually been delivered to the patient. Docking section 330 is configured to accept removable cartridge holder 304. Controller 332 can comprise a microprocessor and/or other appropriate logic control circuits. FIG. 9 depicts a modified cartridge holder 340 that has a hand piece support comprising a first element 342 and a second element 344 such that hand piece 308 can be conveniently supported when not in use. It is convenient to have a hand piece support on the cartridge holder since contact with the hand piece after use can result in contamination with bodily fluid and since the cartridge holder can be disposed following the procedure. Other designs of the hand piece support can be used as desired. Cartridge holder element 342 may comprise or be connected to a heating element that delivers heat to the handpiece.

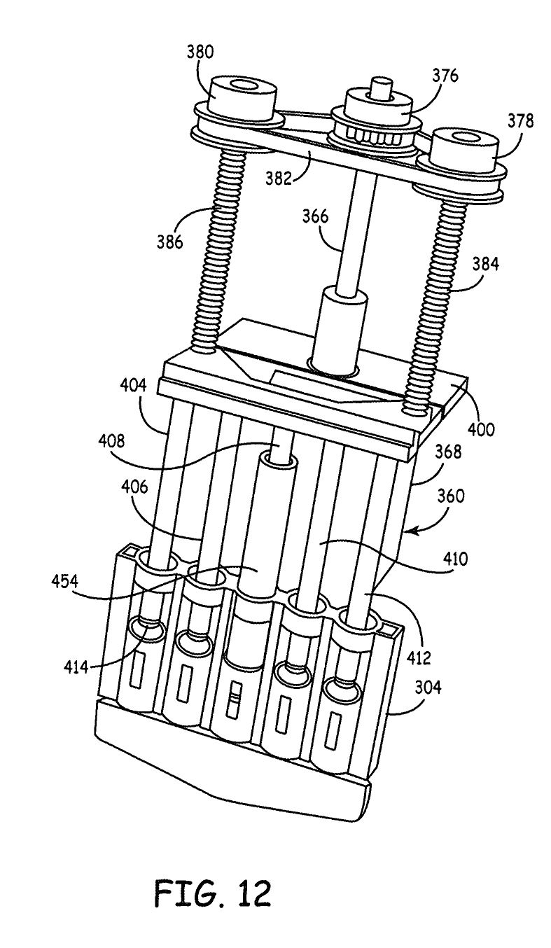

Referring to FIGS. 10-12, drive unit 334 comprises a stepper motor 360, transmission 362, and piston drive 364. Stepper motor 360 comprises a drive shaft 366 extending from motor housing 368. Stepper motor 360 is electrically connected to a transformer 370 to provide power for the motor. Suitable commercial stepper motors and transformers can be used. Transmission 362 comprises a sprocket drive system that has three sprockets 376, 378, 380 with a drive belt 382. Transmission 362 further comprises lead screws 384, 386 that operably connect transmission 362 with piston drive 364. Drive belt 382 transfers motion of drive shaft 366 and associated sprocket 376 with rotation of sprockets 378, 380 and correspondingly lead screws 384, 386. As shown in FIG. 12, sprockets 376, 378, 380 are supported by support plate 390.

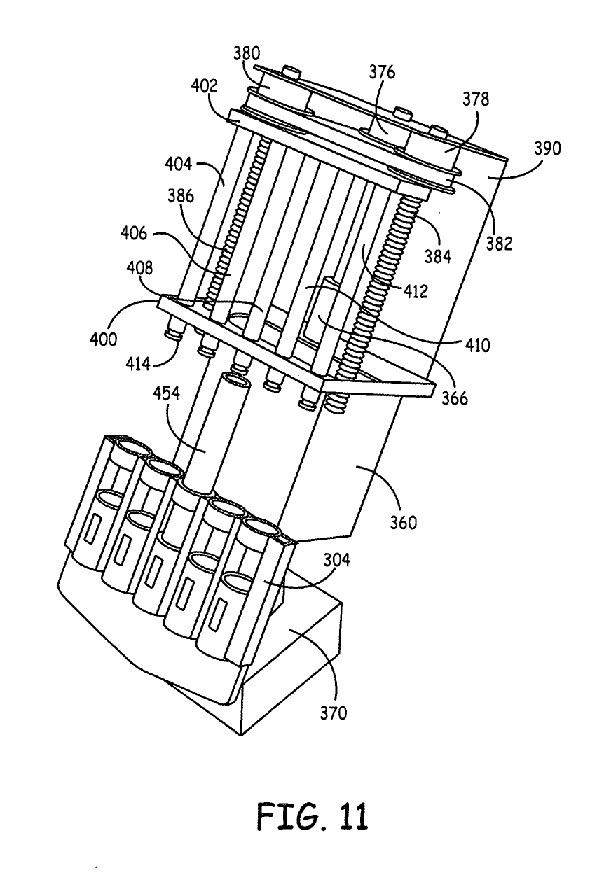

Piston drive 364 comprises a guide/support plate 400, a drive plate 402 and five pistons 404, 406, 408, 410, 412. One end of pistons 404, 406, 408, 410, 412 are fastened to drive plate 402, and the other end of pistons 404, 406, 408, 410, 412 extend through openings in guide/support plate 400 such that pistons are supported by the plate while the pistons can translate through the openings. Lead screws 384, 386 are attached to guide/support plate 400 and can freely rotate at the connection to plate 400. In contrast, lead screws 384, 386 have a threaded connection with drive plate 402 such that rotation of lead screws 384, 386 translates drive plate 402 relative to fixed guide/support plate 400 such that pistons 404, 406, 408, 410, 412 translate through the holes in guide/support plate 400. As shown in FIGS. 10 and 11, pistons 404, 406, 408, 410, 412 are in their fully withdrawn configuration while in FIG. 12, pistons 404, 406, 408, 410, 412 are in their fully inserted position. The motor generally is controlled to stop when the pistons reach their end points in either the forward or reverse directions.

Pistons 404, 406, 408, 410, 412 each comprise an o-ring 414 near the end of the piston, although other type seal elements, such as harpoons and screws, can be used as an alternative to the o-ring. O-ring 414 has an appropriate size to insert into a cartridge with a tight seal. Thus, as the o-ring is advanced or withdrawn, a plug in the cartridge moves correspondingly due to the sealed conditions between the plug and the o-ring without direct physical engagement of the piston with the cartridge plug. The use of a seal on the piston has the advantage over a bur, harpoon, hook or the like that physically engages the cartridge plug since with the use of a o-ring or seal full withdrawal of the piston does not result in a risk of withdrawing the plug from the cartridge tube, which could subject the based unit to contamination from a patient's bodily fluids.

Referring to FIGS. 7 and 8, removable cartridge holder 304 comprises five slots 420, 422, 424, 426, 428 accessed respectively through openings 430, 432, 434, 436, 438. Each slot holds a cartridge to provide fluid delivery. Cartridge holder 304 further comprises wings 450, 452 to facilitate holding cartridge holder 304 during placement and withdrawal of cartridge holder 304 from docking section 330. Cartridge holder 304 further comprises an output port 354 in fluid communication with a manifold, described further below. Output port 354 is fluidly connected to flexible tubing 306. Suitable characteristics for flexible tubing 306 are comparable to the characteristics of flexible tubing 108, described above with respect to FIGS. 1 and 5. FIGS. 10-12 depict a fragmentary view of cartridge holder 304 positioned adjacent drive unit 334 with one cartridge 454 in position.

An exploded view of cartridge holder 304 is shown in FIG. 13. In this embodiments, cartridge holder 304 further comprises a manifold 460 embedded within base 461, where manifold 460 is in fluid communication with transfer tubes 462, 464, 466, 468, 470. The transfer tubes are aligned to engage a cartridge loaded into slots 420, 422, 424, 426, 428, respectively. As shown in this embodiment, tip covers 480, 482, 484 cover the sharpened tip of transfer tubes 462, 464, 466, although in some embodiments tip covers cover all the transfer tubes. The tip covers are designed to form a fluid tight seal over the tip of the transfer tubes as supplied. However, when engaged by a cartridge during the loading process, the tip cover collapses and the sharpened tip of the transfer tube punctures through the cartridge's puncturable cap to establish a fluid connection between the transfer tube and the cartridge. As shown in FIG. 13, a first cartridge 490 is partially inserted into slot 422, and a second cartridge 492 is in position for insertion into slot 420.

Referring to FIGS. 7 and 8, hand piece 308 has a fluid connection with flexible tubing 306. Hand piece 308 comprises a shield segment 500 and a delivery segment 502. Shield segment 500 moves relative to delivery segment 502 with a spring to control this movement with the unbiased position being a closed configuration shown in FIG. 8 to reduce the risk of accidental needle pricks. Delivery segment 502 comprises connection 504 to connect to flexible tubing 306. Delivery segment 502 further comprises finger hold 506 and needle 508 with a fluid connection between tubing connection 504 and needle 508. Shield segment 500 comprises grip 510. FIG. 7 depicts hand piece 308 in a delivery configuration with needle 508 exposed for use. The overall design of hand piece 308 mimics the grip and hand alignment of a conventional dental syringe so that an experienced dentist can efficiently transition to the use of hand piece 308 with a desirable comfort level, and can comfortably move shield element 500 from the safe position, with the needle covered, to the ready position (as shown in FIG. 7) and back to the safe position when the injection is finished.

As shown in FIGS. 1 and 7, slots for different cartridges have the same shape, size and configuration. However, in some embodiments, it can be desirable to have different slots having different shapes, sizes or configuration such that cartridges with different contents generally cannot be loaded in inappropriate slots. Thus, for example, for dental applications, it may be desirable for anesthetic cartridges to have a different size or shape from a cartridge of bicarbonate or other base. Then, one slot would generally have the size and shape for the base while the remaining slots would have the size and configuration for the anesthetic cartridges.

Delivery Procedure

With respect to the approaches for delivering drugs described herein, the procedures provide for the convenient and efficient delivery of drugs from one or more sealed cartridges to a patient. In some embodiments, the heating of the drug can provide a more desirable outcome, such as through the reduction of pain associated with the delivery process and/or increased efficacy of the drug. In alternative or additional embodiments, the contents of a plurality of cartridges can be delivered efficiently without reloading to provide a desired amount of a drug greater than held by a single cartridge and/or to provide a plurality of different compositions simultaneously or sequentially. These procedures are particularly advantageous for administration of dental anesthetics.

As noted above, the procedures described herein can be advantageously used for the administration of a range of drugs to a patient in various forms. In some embodiments, the administration can be hypodermic, such as with a needle. A needle-less jet injector for hypodermic administration is described in U.S. Pat. No. 6,689,093 to Landau, entitled “Single-Use Needle-Less Hypodermic Jet Injection Apparatus and Method,” incorporated herein by reference. A needless injector can be incorporated into the delivery component of the apparatuses described herein.

The procedure generally comprises loading selected cartridges, programming the unit for desired parameters, instructing the instrument to initiate the procedure, optionally priming the system, optionally have an initial delivery period followed by an aspiration period and then provide the drug in one or more delivery steps. The loading of the cartridges may or may not involve breaking the seal on the cartridge. For example, the seal can be broken at a later stage during the process when the cartridge is to be used. If the seal is not broken when loaded, the operator can decide later that the cartridge is not needed and then the cartridge is not wasted. This is advantageous particularly for embodiments in which the cartridges can be delivered sequentially rather than simultaneously. In some embodiments, the cartridge can be visually distinguishable, such as color coded, or its size, its shape or other feature or combination of features can indicate the contents of the cartridge to the practitioner and/or the system. In some embodiments, the system may use this information to create desirable delivery characteristics and/or to deliver a desired mixture of drugs. In some embodiments, these features may prevent certain cartridges from being used together, or in a number that exceeds a desired value. In some embodiments, the operator can select a desired dosage for each delivery, the flow rate during delivery and/or other parameters.

The initiation of a procedure can be controlled with a positionable switch and/or a switch mounted on the housing. In some embodiments, the system can prime the delivery components with a delivery rate selected to purge air from the system and/or to provide for the initial placement of the delivery component, for example, at a rate appropriate to limit pain during the placement of a hypodermic needle into tissue. In some embodiments where delivery of the drug into a blood vessel is undesirable, this initial step can be followed by an aspiration step in which fluid is withdrawn from the patient to verify that the tip of the hypodermic needle is not located in a blood vessel. If it is not, the drug can be delivered at a selected rate. In some embodiments, the system steps through the phases of the of the delivery process automatically at prescribed intervals, while in other embodiments a switch or the like is used to transition between steps.

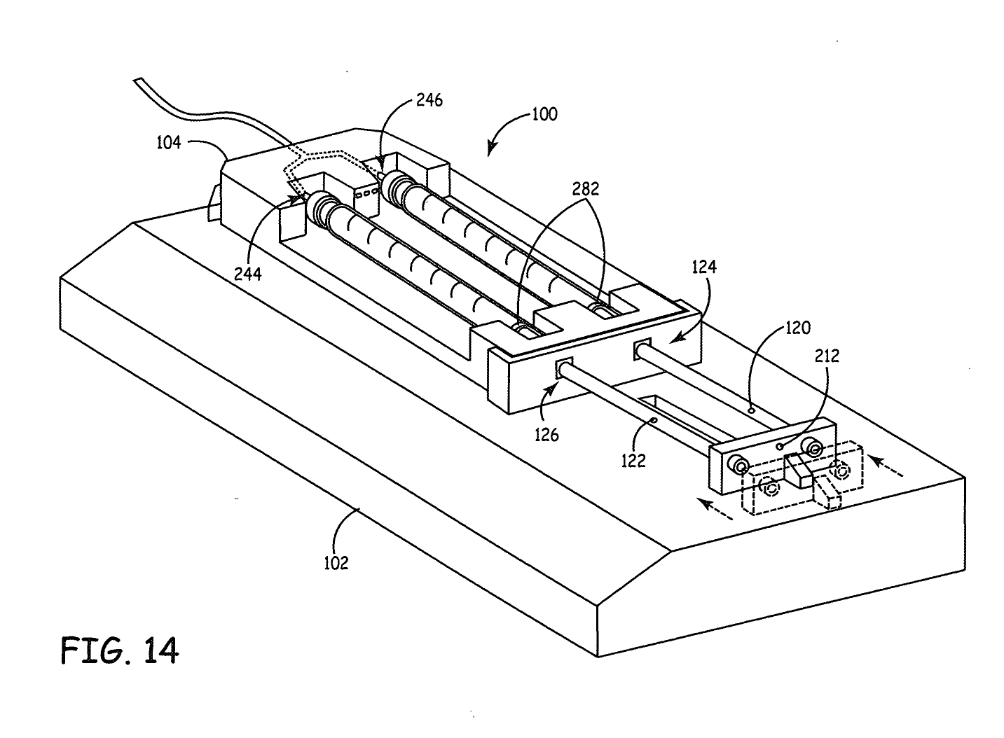

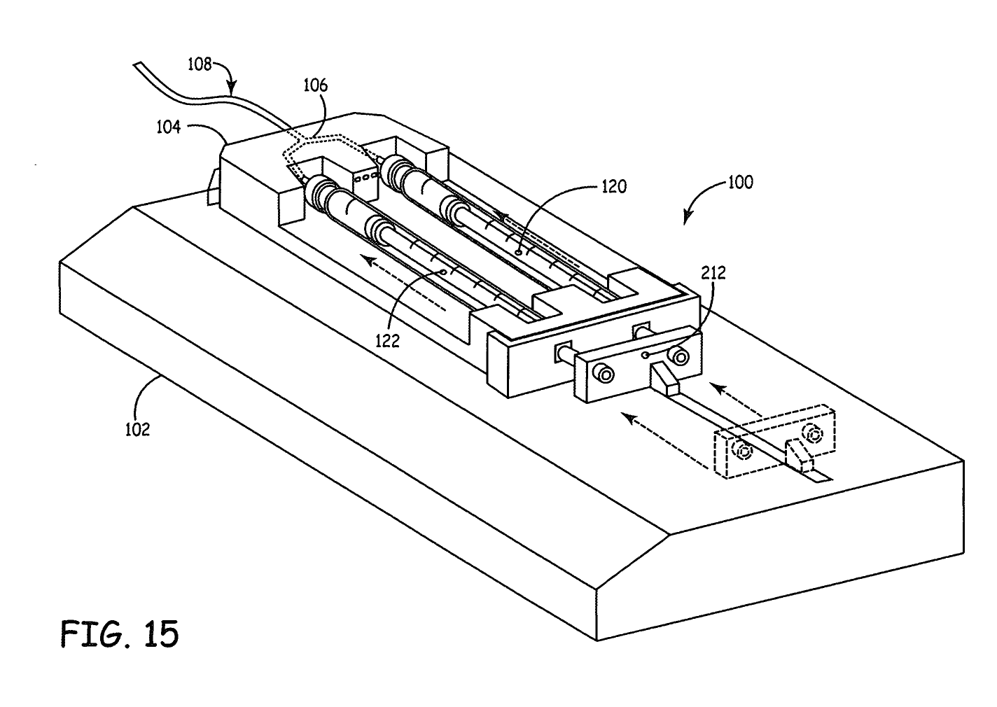

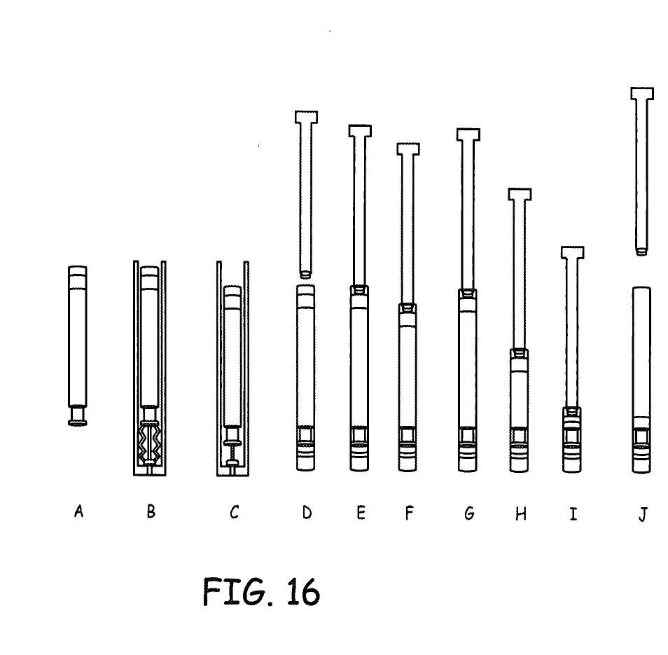

FIGS. 14 and 15 display delivery system 100 of FIGS. 1-6 at two stages of delivery process. As shown in FIGS. 1 and 2, the cartridges are loaded, but the procedure has not yet been initiated. Referring to FIG. 14, delivery system 100 is shown in the priming phase with plate 212 advanced a small distance to correspondingly move slidably mounted plugs 282 in the cartridges to force a small amount of liquid through the other delivery components to purge air from the system. Referring to FIG. 15, delivery system 100 is shown in a later stage of delivery of the drug. As shown in FIG. 15, plate 212 has advanced further, pistons 120, 122 have advanced a majority of their distance within the cartridges so that a corresponding majority of the drug from the cartridges has been delivered through manifold 106 into flexible tubing 108.

Referring to FIG. 16, a cartridge is shown relative to a slot of cartridge holder 304 and a piston of piston drive 364 of the embodiment of delivery system of FIGS. 7 and 8. Specifically, a cartridge is shown in image A. In image B, the cartridge is shown in a slot of cartridge holder 304 prior to puncturing of the seal, situated above the transfer tube with a tip cover attached and sealing the sharpened tip of the transfer tube. Referring to image C, the cartridge has been advanced to compress the tip cover, thus exposing the sharp end of the transfer tube, which punctures and penetrates the cartridge's puncturable cap, to create a fluid connection between contents of the cartridge and the transfer tube. Referring to image D, the piston is shown in position over the cartridge after the transfer tube has punctured the seal of the cartridge of the cartridge, although in alternative embodiments force from the piston, rather than force applied during loading of the cartridge, is used to puncture the seal of the cartridge with the transfer tube. In image E, the piston is shown in contact with the slidably mounted plug. Referring to Fig. F, the piston is depressed to the priming position. As shown in image G, aspiration has been performed through withdrawing the piston. In image H, the piston is shown part way through the delivery process, and in image I, the piston has advanced to its full travel position. Referring to image J, the piston is retracted fully such that the spent cartridge and other components can be appropriately disposed.

For embodiments in which compositions are mixed in the manifold for delivery of the resulting drug, the relative amounts of the compositions can be selected, for example, through the concentration of the individual compositions, the diameter of the different cartridges, which correspondingly changes the cartridge volume, and/or through the delivery rate from the respective cartridge. Apparatus designs for selective delivery rates from different cartridges are discussed above.

For a specific embodiment relating to the delivery of a dental anesthetic using the systems described herein with a cartridge holder that can hold up to five anesthetic cartridges, a representative detail procedure is as follows.

Anethetization Process Steps Using System

1) Open sterile package containing disposables (handpiece and cartridge holder)
2) Insert up to five anesthetic cartridges in cartridge holder, then:
   a. If no buffering desired, skip to Step 3
   b. If buffering required insert buffering cartridge in center slot of cartridge holder go to Step X
3) Load cartridge holder into base unit, then:
   a. If warming not desired, skip to Step 4
   b. If warming desired, turn on warmer and allow system to warm
4) Select anesthetic type, desired dose and delivery speed on base unit (system primes itself when set)
5) Slide safety sheath into ready position
6) Manipulate handpiece into mouth to place tip of needle over the injection site
7) Insert needle into patient's tissue at injection site
8) Step on foot pedal (system aspirates for 2 seconds, then pauses 3 seconds to allow inspection)
9) Inspect aspiration window on handpiece for visible presence of blood (indicating needle is in blood vessel)
   a. If no blood go to step 14
   b. If blood appears,
      i. Step off foot pedal
      ii. Relocate needle and go back to step 11
10) System starts injection automatically after pausing 3 seconds for inspection, continues until:
   a. Injection stops because dentist steps off pedal
   b. Injection stops because pre-set dosage has been delivered
11) Remove needle from tissue
12) Remove syringe from mouth
13) Slide safety sheath closed
14) Wait 10-15 minutes
15) Test patient for numbness
   a. If sufficient anesthetic has been delivered, go to step 16
   b. If more anesthetic needs to be administered, go to Step 5
16) Start dental procedure
   a. If patient remains numb for entire procedure, go to Step 16
   b. If patient regains feeling during procedure, go to Step 5
17) Complete dental procedure
18) Discard handpiece, tube and cartridge holder in garbage.
19) Wipe down base unit with cleaner at end of day.

The embodiments above are intended to be illustrative and not limiting. Additional embodiments are within the inventive concepts. Although the present invention has been described with reference to particular embodiments, workers skilled in the art will recognize that changes may be made in form and detail without departing from the spirit and scope of the invention. The incorporations by reference above of the indicated references are limited to the extent to exclude subject matter that is directly contradictory to the explicit disclosure herein.

What we claim is:

1. A method for the delivery of multiple units of a drug from sealed cartridges to a patient, the method comprising selectively delivering the drug from a plurality of individually sealed first cartridges using a motor to advance a plug within a cartridge to drive the drug from the cartridge, the first cartridges being positioned on a stationary housing, wherein the drug from the first cartridges flows through a manifold fluidly connected to flexible tubing, wherein the flexible tubing is fluidly connected to a delivery component that delivers the drug to the patient and wherein the drug flows from the first cartridges to the delivery component through a sealed fluid path.

2. The method of claim 1 wherein the drug is simultaneously driven from the plurality of first cartridges.

3. The method of claim 1 wherein the delivery component comprises a hypodermic needle and wherein the delivering comprises performing a hypodermic injection.

4. The method of claim 1 wherein the plurality of first cartridges are supported within a cartridge holder and wherein the manifold is embedded within the cartridge holder.

5. The method of claim 1 further comprising aspirating liquid from the patient through reversing the movement of the plug.

6. The method of claim 1 wherein an at least one second cartridge comprises a composition different from the drug in the first cartridge and wherein the composition is delivered to the delivery component through the manifold.

7. The method of claim 1 further comprising a controller that can be programmed to deliver a volume of the drug to the patient.

8. The method of claim 1 wherein the drug comprises anesthetic, wherein at least one second cartridge comprises an alkaline composition, and wherein a mixture of the drug and the alkaline composition is formed in the manifold to raise the pH of the drug.

9. The method of claim 1 further comprising warming the fluid before it is delivered from the delivery component.

10. A drug delivery system comprising:
a housing comprising at least one motor operably connected to a plurality of pistons;
a cartridge holder comprising a plurality of positioning slots configured to hold a fluid cartridge in an alignment to be engaged by one of the pistons when the piston is translated;
a plurality of transfer tubes comprising a first end configured to engage and enter a sealed cartridge at a positioning slot, wherein at least one transfer tube comprises a displaceable cap covering the first end of the transfer tube;
a manifold comprising an output port and a plurality of channels wherein a channel is fluidly connected with a transfer tube and wherein the manifold has a configuration in which flows from the channels combine while having a fluid connection to the output port;
flexible tubing comprising a first end and a second end, wherein the first end is fluidly connected to the output port of the manifold; and
a patient delivery component comprising a fluid outlet, the patient delivery component being fluidly connected with the second end of the flexible tubing.

11. The drug delivery system of claim 10 wherein the manifold is embedded within the cartridge holder.

12. The drug delivery system of claim 10 wherein the transfer tubes are integral with the manifold.

13. The drug delivery system of claim 10 wherein the cartridge holder is releasably engaged with the housing.

14. The drug delivery system of claim 10 further comprising a controller comprising input controls wherein the controller is operably connected to the at least one motor to control the function of the at least one motor and wherein the at least one motor is operably connected to a drive that simultaneously propels the pistons.

15. The drug delivery system of claim 10 further comprising a controller comprising input controls wherein the controller is operably connected to the at least one motor to control the function of the at least one motor and wherein the at least one motor is operably connected to a drive that propels the pistons in a selected pattern wherein the pistons are not driven at equal rates.

16. The drug delivery system of claim 15 wherein the at least one motor comprises at least two motors that drive different pistons.

17. The drug delivery system of claim 16 wherein the at least two motors have adjustable speeds to provide the capability to propel the different pistons at different speeds.

18. The drug delivery system of claim 10 further comprising a plurality of cartridges comprising drugs, wherein each cartridge is loaded at a positioning slot.

19. The drug delivery system of claim 10 further comprising a plurality of cartridges comprising anesthetic, wherein each cartridge is loaded at a positioning slot.

20. The drug delivery system of claim 10 wherein the piston is configured to move a plug within a cartridge in a forward or reverse direction and wherein the controller is programmed to withdraw the piston a select amount to provide for aspiration.

21. The drug delivery system of claim 10 further comprising at least one heating element configured to heat at least one component of the system.

22. The drug delivery system of claim 10 further comprising a plurality of cartridges wherein at least one cartridge comprises a first drug and at least one cartridge comprises a second drug.

23. The drug delivery system of claim 10 further comprising a plurality of cartridges wherein at least one cartridge comprises an anesthetic and at least one cartridge comprises an alkalinizing composition.

24. A drug delivery system comprising:
a housing comprising at least one motor operably connected to at least one piston;
a cartridge holder having at least one positioning slot configured to hold a cartridge;
at least one transfer tube configured to enter a sealed cartridge to establish a flow passage to an output port;
flexible tubing having a first end and a second end wherein the first end if fluidly connected to the output port;
a patient delivery component comprising a fluid outlet, the patient delivery component being fluidly connected with the second end of the flexible tubing; and
a heating element, wherein the heating element is configured to heat at least one component of the system.

25. The drug delivery system of claim 24 wherein the housing comprises a plurality of positioning slots wherein each positioning slot is configured to hold a drug cartridge for the delivery of drugs from the cartridge.

26. The drug delivery system of claim 24 wherein the cartridge holder is removably connected to the housing.

27. A method for the delivery of a mixture of drugs to a patient, the method comprising delivering a mixture of different drugs from a manifold connected to a plurality of cartridges comprising at least a first cartridge and a second cartridge, which cartridges contain different drugs and are positioned on a stationary housing, using a motor to move a plug within the first cartridge to drive the drug from the first cartridge to the manifold wherein the manifold is fluidly connected to flexible tubing and wherein the flexible tubing is fluidly connected to a delivery component to deliver the mixture.

28. The method of claim 27 wherein the motor moves a plug within the second cartridge to drive the drug from the second cartridge to the manifold.

29. The method of claim 27 further comprising a second motor that moves a plug in the second cartridge to drive the drug from the second cartridge to the manifold.

30. The method of claim 27 wherein the first cartridge comprises an anesthetic and the second cartridge comprises an alkalinizing substance.

31. The method of claim 30 further comprising a second motor that moves a plug in the second cartridge positioned on the stationary housing to drive the alkalinizing substance from the second cartridge to the manifold at a selectable rate to achieve a desired pH of the mixture.
